(19) United States
(12) Patent Application Publication
(10) Pub. No.: US 2010/0198064 A1
(43) Pub. Date: Aug. 5, 2010

(54) DEVICES AND METHODS FOR NON-INVASIVE ULTRASOUND-GUIDED BODY CONTOURING USING SKIN CONTACT COOLING

(76) Inventors: Paul K. Perl, Givatiyim (IL); Francisco Arriaza Munoz, Barcelona (ES)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 11/916,675

(22) PCT Filed: Nov. 25, 2007
(86) PCT No.: PCT/IL07/01450

§ 371 Date: Dec. 6, 2007

(30) Foreign Application Priority Data
Feb. 16, 2007 (ES) 1064835U
Feb. 16, 2007 (ES) 1064836U

(51) Int. Cl. A61B 8/00; A61N 7/00; A61N 1/04

(52) U.S. Cl. 600/439; 607/115

(57) ABSTRACT

The present invention discloses devices and methods, for non-invasive ultrasound-guided body contouring, including: a variable-frequency treatment applicator having at least one variable-frequency ultrasound emitter; and a control unit for adjusting an output frequency of at least one ultrasound emitter. Devices and methods including: a variable-frequency treatment applicator having at least one variable-frequency ultrasound emitter; a resonance sensor for determining a resonant frequency of a treatment area; and a control unit for adjusting an output frequency, of at least one ultrasound emitter, to the resonant frequency based on a signal from the resonance sensor. Devices and methods including: a variable-frequency treatment applicator having at least one variable-frequency ultrasound emitter; a cooling mechanism located in the treatment applicator; and a control unit for applying an output frequency to at least one ultrasound emitter. Preferably, the output frequency is within a frequency range from 25 kHz to 60 kHz.

This patent application claims priority under 35 U.S.C. §119(e) to Spanish Utility Model Patent Application Nos. ES1064835U and ES1064836U, filed Feb. 16, 2007, which are hereby incorporated by reference in their entirety.

### FIELD AND BACKGROUND OF THE INVENTION

The present invention relates to devices and methods for non-invasive ultrasound-guided body contouring using skin contact cooling for use in medical therapies and cosmetic treatments for the human body by lysing adipose tissue.

In the prior art, there is a wide range of useful devices for medical and cosmetic treatments that use different types of energy to obtain beneficial effects. For example, Eshel, U.S. Pat. No. 6,607,498 (hereinafter referred to as Eshel '498), teaches a device having a directional head, including one or more ultrasound emitters, that can produce beneficial vibrational and cavitational effects in a patient's superficial and internal tissues.

In Eshel '498, focused ultrasound energy is administered at a pre-determined power and frequency that can be adjusted from a control unit connected to the device head. The adjustment is performed manually according to the treatment to be administered and the area to be treated. In such an arrangement, the appropriate energy to be applied is typically determined by the operator or therapist, and thus, depends on their expertise and experience. The effect of the ultrasound therapy on lysing adipose tissue is not known at the time of treatment.

Due to the configuration of tissue and organs in the human body, there is a resonant frequency at which the applied energy is more effective and better absorbed. However, devices known in the art for such treatment do not provide a way to effectively determine the resonant frequency.

Determining the resonant frequency enables the operator or therapist to optimize the treatment results to the patient during treatment, to avoid excessive exposure to the patient of unutilized energy, and to prevent harmful side effects that can result from inappropriate treatment conditions. There is a risk that the applied power may be too high to produce a given effect. This can result in local inflammation due to excessive cavitation or overheating by friction. Current methods attempt to avoid such situations from occurring by treating the patient on a frequent basis in short sessions, inconveniencing the patient by wasting time in making multiple visits with partial results.

It would be desirable to have devices and methods for non-invasively lysing adipose tissue, as described above, in which the treatment is performed using optimal parameters based on the appropriate resonant frequency for the patient.

### SUMMARY OF THE INVENTION

It is the purpose of the present invention to provide devices and methods for non-invasive ultrasound-guided body contouring using skin contact cooling.

For the purpose of clarity, the term “variable-frequency treatment applicator” is specifically defined for use herein to refer to an applicator that can output a frequency that is continuously variable over a frequency range, meaning that the output frequency that the applicator emits is continuously variable in real time.

Embodiments of the present invention use a treatment applicator having one or more variable-frequency ultrasound emitters to adjust the output energy, either automatically or manually, to the resonant frequency detected for each patient via a resonance sensor.

In preferred embodiments of the present invention, the treatment applicator includes a low- and mid-frequency electro-stimulation electrode.

In other preferred embodiments of the present invention, the treatment applicator includes at least one insulated high-frequency stimulation electrode, either resistive or capacitive.

In preferred embodiments of the present invention, the device includes a resonance sensor of the energy administered by the ultrasound emitters, allowing for the measurement and evaluation of the amount of absorbed and reflected energy. The resonance sensor is connected to a control module to determine the working frequency that provides the highest efficiency of power with the patient's tissue.

In preferred embodiments of the present invention, the device scans the entire working frequency range, and measures the frequency at which the supplied ultrasound is most efficient (via the resonance sensor). The optimal frequency corresponds to the resonant frequency of the energy applied to the tissue in the specific area of the patient's anatomy, and ensures better therapeutic results, while reducing exposure to unutilized energy.

In another preferred embodiment of the present invention, the resonance sensor may be located in a separate device head, independent of the treatment applicator in which the ultrasound emitters are located.

Therefore, according to the present invention, there is provided for the first time a device for non-invasive ultrasound-guided body contouring, the device including: (a) a variable-frequency treatment applicator having at least one variable-frequency ultrasound emitter; and (b) a control unit for adjusting an output frequency of at least one ultrasound emitter.

Preferably, the treatment applicator has at least two ultrasound emitters configured to be operated sequentially.

Preferably, the output frequency is within a frequency range from 20 kHz to 100 kHz.

Preferably, the output frequency is within a frequency range from 25 kHz to 60 kHz.

Preferably, the control unit is configured to provide the output frequency in a continuous-wave mode.

Preferably, the control unit is configured to provide the output frequency in a burst-cycle mode.

Preferably, the control unit is configured to sweep the output frequency over a designated frequency range and a designated time interval.

Preferably, the treatment applicator includes at least one electro-stimulation electrode.

According to the present invention, there is provided for the first time a device for non-invasive ultrasound-guided body contouring, the device including: (a) a variable-frequency treatment applicator having at least one variable-frequency ultrasound emitter; (b) a resonance sensor for determining a resonant frequency of a treatment area; and (c) a control unit for adjusting an output frequency, of at least one ultrasound emitter, to the resonant frequency based on a signal from the resonance sensor.

Preferably, the resonance sensor is located in the treatment applicator.

Preferably, the resonance sensor is located in a separate head independent of the treatment applicator.

Preferably, the output frequency is within a frequency range from 25 kHz to 60 kHz.

Preferably, the control unit is configured to provide the output frequency in a continuous-wave mode.

Preferably, the control unit is configured to provide the output frequency in a burst-cycle mode.

Preferably, the control unit is configured to sweep the output frequency over a designated frequency range and a designated time interval.

Preferably, the treatment applicator includes at least one electro-stimulation electrode.

According to the present invention, there is provided for the first time a device for non-invasive ultrasound-guided body contouring using skin contact cooling, the device including: (a) a variable-frequency treatment applicator having at least one variable-frequency ultrasound emitter; (b) a cooling mechanism located in the treatment applicator; and (c) a control unit for applying an output frequency to at least one ultrasound emitter.

Preferably, the cooling mechanism is configured to pass a coolant through at least one channel in at least one ultrasound emitter.

Preferably, the cooling mechanism is configured to be controlled by an thermo-electric cooler.

According to the present invention, there is provided for the first time a method for non-invasive ultrasound-guided body contouring, the method including the steps of: (a) providing a variable-frequency treatment applicator having at least one variable-frequency ultrasound emitter; and (b) adjusting, using a control unit, an output frequency of at least one ultrasound emitter.

According to the present invention, there is provided for the first time a method for non-invasive ultrasound-guided body contouring, the method including the steps of: (a) providing a variable-frequency treatment applicator having at least one variable-frequency ultrasound emitter; (b) determining, using a resonance sensor, a resonant frequency of a treatment area; and (c) adjusting, using a control unit, an output frequency, of at least one ultrasound emitter, to the resonant frequency based on a signal from the resonance sensor.

According to the present invention, there is provided for the first time a method for non-invasive ultrasound-guided body contouring using skin contact cooling, the method including the steps of (a) providing a variable-frequency treatment applicator having at least one variable-frequency ultrasound emitter; (b) cooling at least one ultrasound emitter; and (c) applying, using a control unit, an output frequency of at least one ultrasound emitter.

These and further embodiments will be apparent from the detailed description and examples that follow.

### BRIEF DESCRIPTION OF THE DRAWINGS

The present invention is herein described, by way of example only, with reference to the accompanying drawings, wherein:

FIG. 1 shows a perspective view of the ultrasound-guided body-contouring device, according to preferred embodiments of the present invention;

FIG. 2A shows a partial cut-away view of the treatment applicator of the device, according to preferred embodiments of the present invention;

FIG. 2B shows an end view of the skin-contacting surface of the treatment applicator of FIG. 2A, according to preferred embodiments of the present invention.

### DESCRIPTION OF THE PREFERRED EMBODIMENTS

The present invention relates to devices and methods for non-invasive ultrasound-guided body contouring using skin contact cooling. The principles and operation for non-invasive ultrasound-guided body contouring using skin contact cooling, according to the present invention, may be better understood with reference to the accompanying description and the drawings.

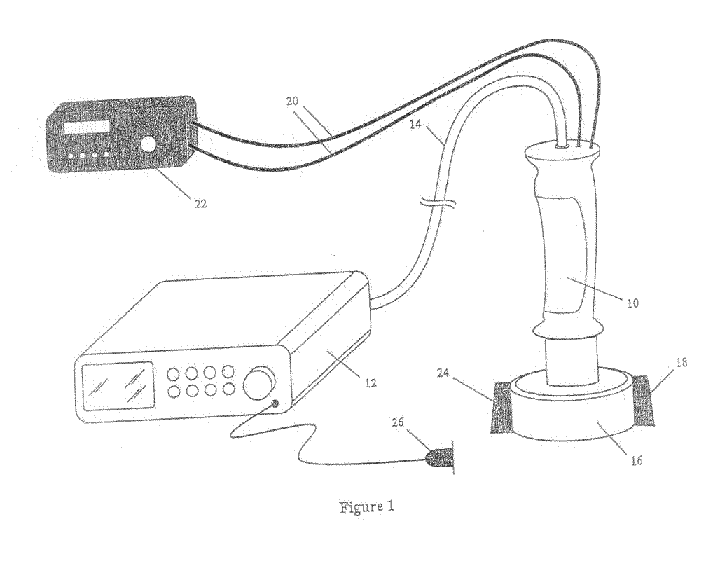

Referring now to the drawings, FIG. 1 shows a perspective view of the ultrasound-guided body-contouring device, according to preferred embodiments of the present invention. A treatment applicator 10 is connected to a control unit 12 via a connection cable 14. An ultrasound emitter 16 (e.g. a piezoelectric element) is positioned at the end of treatment applicator 10. Control unit 12 can be used to sweep the output frequency of ultrasound emitter 16 over a pre-determined range of frequencies.

In preferred embodiments of the present invention, a resonance sensor 18 (e.g. using ultrasound-imaging or impedance-measurement techniques) is connected to control unit 12, and is used to regulate the output frequency and power of ultrasound emitter 16. During a sweep of the output frequency of ultrasound emitter 16 by control unit 12, resonance sensor 18 determines the resonant frequency. Control unit 12 uses the resonant frequency as the working frequency for ultrasound emitter 16, optimizing treatment with minimum power. Alternatively, control unit 12 can also continue to sweep the output frequency of ultrasound emitter 16 in a narrow range centered on the resonant frequency. In other preferred embodiments, resonance sensor 18 is located in a head (not shown) that is independent of treatment applicator 10.

In preferred embodiments of the present invention, control unit 12 is configured such that treatment applicator 10 delivers ultrasonic emission over a wide range of frequencies (e.g. 20-500 kHz). In preferred embodiments, a working frequency range of ultrasonic emission from 25 to 60 kHz is employed. Control unit 12 activates and controls a single piezoelectric element (i.e. ultrasound emitter 16) to provide ultrasound emission.

Ultrasound emitter 16 can be operated in sweeping- or resonant-frequency mode, as well as in a continuous-wave or burst-cycle mode. In the sweeping-frequency mode, a frequency range is chosen, and control unit 12 constantly changes the frequency at pre-determined time intervals continuously. The sweeping-frequency mode enables the depth of treatment to be controlled.

In the resonant-frequency mode, the frequency is fixed at the determined resonant frequency which depends on the volume, density, and depth of the fat tissue being treated in order to produce effective cavitational bubbles. In order to optimize the effectiveness of the treatment, the resonant frequency associated with the fat tissue has to be determined that does not cause an effect on the surrounding tissue.

In the continuous-wave mode, ultrasound emission is applied to the treatment area continuously. Due to the presence of a cooling mechanism (described in greater detail below) in treatment applicator 10, single-treatment sessions can be performed. In the burst-cycle mode, control unit 12 operates in an on/off duty cycle to provide a variety of treatment pulses in order to create a greater amount of micro-bubbles. Furthermore, such burst-mode operation can create shock waves due to localized pressure gradients, enhancing the effectiveness of the treatment.

Resonant absorption of the ultrasound emission depends on the cavity size of the tissue being treated, the density of the tissue, and the depth of the tissue. The resonant frequency is determined manually or automatically by control unit 12 using the data signal from resonant sensor 18 in treatment applicator 10. The micro-bubbles created in the fat tissue, due to the exposure to the ultrasound emission, lyse the adipose tissue due to pressure changes when expanding and collapsing (due to both micro-jet and heating effect below the skin surface with no undesirable heating effect at the skin-contact surface).

In preferred embodiments of the present invention, a coolant circulating in cooling lines 20 is used to dissipate the heat generated by ultrasound emitter 16 in a skin-contact cooling-mode via a thermo-electric cooler 22. Thermo-electric cooler 22 is connected to treatment applicator 10 via cooling lines 20 to supply the cooling at all times to the circulating chamber of ultrasound emitter 16. Such cooling is especially important when the device is operating at non-resonant frequencies and/or in continuous-wave mode.

In other preferred embodiments, an electro-stimulation electrode 24 is mounted on treatment applicator 10 for providing enhanced treatment capabilities. Electro-stimulation electrode 24 applies a low- to mid-frequency (e.g. 5 to 500 Hz) current in order to stimulate and contract the tissue in order to enhance the cavitational effect. Electro-stimulation electrode 24 can also be configured to supply a current in the RF frequency range (e.g. 1 to 10 MHz) in an electrically-isolated probe. During operation, a counter electrode 26 is placed in contact with the patient's body to complete the circuit.

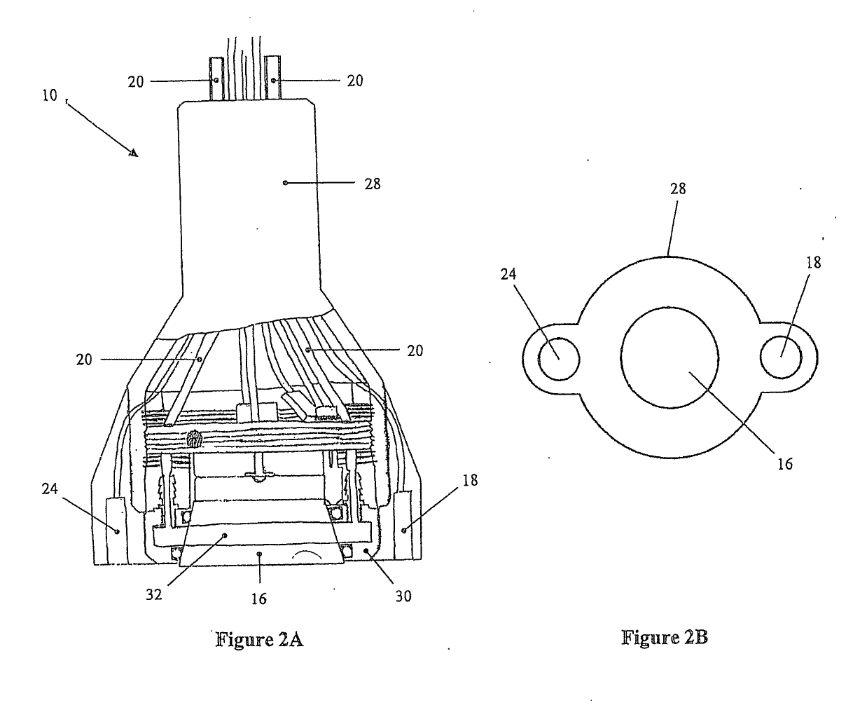

FIG. 2A shows a partial cut-away view of the treatment applicator of the device, according to preferred embodiments of the present invention. In preferred embodiments, treatment applicator 10 is configured to provide localized treatments. Treatment applicator 10 is shown in FIG. 2A with a lower portion of a housing 28 removed to reveal the internal components of treatment applicator 10.

Thermo-electric cooler 22, via cooling lines 20, provides cooling, which can be regulated for a desired temperature, to ultrasound emitter 16 via a circulating jacket 30. Circulating jacket 30 is preferably made of aluminum or another light thermally-conductive material. Coolant, flowing through cooling lines 20, flows through a cooling channel 32 in ultrasound emitter 16. Cooling the piezoelectric element of ultrasound emitter 16 is necessary in order to prevent overheating (while operating at non-resonant frequencies and/or in continuous-wave mode), to provide comfort to the patient, and to allow continuous operation during treatment without interruptions due to “cool-down” periods. During the sweeping of the frequency, the piezoelectric element produces a considerable amount of heat.

FIG. 2B shows an end view of the skin-contacting surface of the treatment applicator of FIG. 2A, according to preferred embodiments of the present invention. Resonance sensor 18 and electro-stimulation electrode 24 are shown within housing 28 outside the region of ultrasound emitter 16.

While the invention has been described with respect to a limited number of embodiments, it will be appreciated that many variations, modifications, and other applications of the invention may be made.

What is claimed is:

1. A device for non-invasive ultrasound-guided body contouring, the device comprising:
(a) a variable-frequency treatment applicator having at least one variable-frequency ultrasound emitter; and
(b) a control unit for adjusting an output frequency of said at least one ultrasound emitter.

2. The device of claim 1, wherein said treatment applicator has at least two ultrasound emitters configured to be operated sequentially.

3. The device of claim 1, wherein said output frequency is within a frequency range from 20 kHz to 100 kHz.

4. The device of claim 1, wherein said output frequency is within a frequency range from 25 kHz to 60 kHz.

5. The device of claim 1, wherein said control unit is configured to provide said output frequency in a continuous-wave mode.

6. The device of claim 1, wherein said control unit is configured to provide said output frequency in a burst-cycle mode.

7. The device of claim 1, wherein said control unit is configured to sweep said output frequency over a designated frequency range and a designated time interval.

8. The device of claim 1, wherein said treatment applicator includes at least one electro-stimulation electrode.

9. A device for non-invasive ultrasound-guided body contouring, the device comprising:
(a) a variable-frequency treatment applicator having at least one variable-frequency ultrasound emitter;
(b) a resonance sensor for determining a resonant frequency of a treatment area; and
(c) a control unit for adjusting an output frequency, of said at least one ultrasound emitter, to said resonant frequency based on a signal from said resonance sensor.

10. The device of claim 9, wherein said resonance sensor is located in said treatment applicator.

11. The device of claim 9, wherein said resonance sensor is located in a separate head independent of said treatment applicator.

12. The device of claim 9, wherein said output frequency is within a frequency range from 25 kHz to 60 kHz.

13. The device of claim 9, wherein said control unit is configured to provide said output frequency in a continuous-wave mode.

14. The device of claim 9, wherein said control unit is configured to provide said output frequency in a burst-cycle mode.

15. The device of claim 9, wherein said control unit is configured to sweep said output frequency over a designated frequency range and a designated time interval.

16. The device of claim 9, wherein said treatment applicator includes at least one electro-stimulation electrode.

17. A device for non-invasive ultrasound-guided body contouring using skin contact cooling, the device comprising:
(a) a variable-frequency treatment applicator having at least one variable-frequency ultrasound emitter;
(b) a cooling mechanism located in said treatment applicator; and
(c) a control unit for applying an output frequency to said at least one ultrasound emitter.

18. The device of claim 17, wherein said cooling mechanism is configured to pass a coolant through at least one channel in said at least one ultrasound emitter.

19. The device of claim 17, wherein said cooling mechanism is configured to be controlled by a thermo-electric cooler.

20. A method for non-invasive ultrasound-guided body contouring, the method comprising the steps of:
(a) providing a variable-frequency treatment applicator having at least one variable-frequency ultrasound emitter; and
(b) adjusting, using a control unit, an output frequency of said at least one ultrasound emitter.

21. A method for non-invasive ultrasound-guided body contouring, the method comprising the steps of:
(a) providing a variable-frequency treatment applicator having at least one variable-frequency ultrasound emitter;
(b) determining, using a resonance sensor, a resonant frequency of a treatment area; and
(c) adjusting, using a control unit, an output frequency, of said at least one ultrasound emitter, to said resonant frequency based on a signal from said resonance sensor.

22. A method for non-invasive ultrasound-guided body contouring using skin contact cooling, the method comprising the steps of:
(a) providing a variable-frequency treatment applicator having at least one variable-frequency ultrasound emitter;
(b) cooling said at least one ultrasound emitter; and
(c) applying, using a control unit, an output frequency of said at least one ultrasound emitter.
